(19) United States
(12) Patent Application Publication
(10) Pub. No.: US 2013/0236881 A1
(43) Pub. Date: Sep. 12, 2013

(54) THREE-DIMENSIONAL METAL-COATED NANOSTRUCTURES ON SUBSTRATE SURFACES, METHOD FOR PRODUCING SAME AND USE THEREOF

(71) Applicants: Joachim P. Spatz, Stuttgart (DE); Claudia Pacholski, Stuttgart (DE); Tobias Schoen, Stuttgart (DE); Lindarti Purwaningsih, Stuttgart (DE); Tobias Wolfram, Dreieich (DE)

(72) Inventors: Joachim P. Spatz, Stuttgart (DE); Claudia Pacholski, Stuttgart (DE); Tobias Schoen, Stuttgart (DE); Lindarti Purwaningsih, Stuttgart (DE); Tobias Wolfram, Dreieich (DE)

(73) Assignee: Max-Planck-Gesellschaft zur Foerderung der Wissensdchaften e.V., Muenchen (DE)

(21) Appl. No.: 13/703,054

(22) PCT Filed: May 30, 2011
(86) PCT No.: PCT/EP2011/002670

§ 371 Date: May 7, 2013

(30) Foreign Application Priority Data
Jun. 11, 2010 (DE) 10 2010 023 490.7

(51) Int. Cl. G01N 33/553; A61B 5/00; A61B 5/145

(52) U.S. Cl. CPC G01N 33/553; A61B 5/14503; A61B 5/6848
USPC 435/5; 435/7.21; 435/7.1; 436/501; 216/11; 600/309

(57) ABSTRACT

The invention relates to a method for producing column-shaped or conical nanostructures, wherein the substrate surface is covered with an arrangement of metal nanoparticles and etched, the nanoparticles acting as an etching mask and the etching parameters being set such that column structures or cone structures are created below the nanoparticles and the nanoparticles are preserved as a structural coating.

Three-dimensionally nanostructured substrate surfaces, which can be functionalized with binding molecules to enable the selective binding of biological structures and molecules, in particular cells, are in principle known in the prior art. Nagrath et al. describe in Nature, 450, 1235-1239 (2007), the production of surfaces with column-like structures with a length in the micrometer range for enrichment of circulating tumor cells, and Wang et al. describe in Angew. Chem. Int. Ed., 48, 8970-8973 (2009), the production of Si nanopillars on a Si wafer with the help of a wet-chemical etching method and the functionalization with a specific antibody, Anti-EpCAM, which enables the selective binding of certain tumor cells. However, the production of these nanostructures and also their functionalization is relatively time- and cost-consuming. Structurally, the published structures are within the urn length range (100-200 nm diameter, length 10 μm). Therefore, these structures are not of the ideal size for immobilization of ordered molecule surfaces. Furthermore, in these structures, the number of molecules per surface unit in the urn range is reduced compared to nanostructures. The controlled long-term cultivation and differentiation of cells can not be performed with the published structure functionalization.

A simple and inexpensive method, using which three-dimensional nanostructures for optical elements can be created directly on guartz glass by means of etching, is described in the German laid-open specification DE 10 2007 014 538 A1 and in the corresponding international publication WO 2008/116616 A1, as well as in Lohmuller et al., NANO LETTERS 2008, Vol. 8, No. 5, 1429-1433. However, the nanopillars disclosed there are not metal-coated and functionalization with biological binding molecules is not proposed. These nanopillars of the prior art do not contain any metal particles or metal deposits on their surface after the etching process, because the metal, which was first applied as a mask, is completely vaporized during the etching process. This is absolutely necessary for the functionality of the structures described there as an optical element. A biofunctionalization of these conventional nanostructures would be only possible by means of costly silanization reactions under protective gas atmosphere in the course of many hours (at least 8 h). The published structure does not permit any chemically ordered functionalization with bioactive molecules, because during the silanization the structural integrity of the molecules is lost.

Against this background, an object of the present invention was the provision, in particular for biomedical, bioanalytical and biosensoric applications, of improved three-dimensional nanostructures on a substrate surface, which can be functionalized in a simple way with a plurality of binding molecules and enable the selective binding of biological structures and molecules, as well as cells or cell clusters with high efficiency and yield.

This object is achieved according to the invention with the provision of the method according to claim 1 as well as the substrate surface according to claim 13 and the device according to claim 18. Specific or preferred embodiments and aspects of the invention are the subject matter of the further claims.

### DESCRIPTION OF THE INVENTION

The method according to the invention for generating column-like or conical nanostructures, which have a metal coating on their upper side on a substrate surface according to claim 1 comprises at least the following steps:
a) providing a substrate surface coated with SiO₂ or consisting of SiO₂;
b) covering the substrate surface with an arrangement of metal nanoparticles;
c) contacting the substrate with a metal salt solution under reducing conditions, whereby a reduction of the metal salt and a currentless deposition of elemental metal on the metal nanoparticles, as well as a corresponding growth of the metal nanoparticles is caused;
d) etching of the substrate surface covered with the nanoparticles obtained in step c) in a depth of 10-500 nm, wherein the nanoparticles act as an etching mask and the etching parameters are adjusted in such a way that column-like structures or conical structures are formed underneath the nanoparticles and the nanoparticles remain kept there as a structure coating.

The primary substrate surface is basically not particularly limited and may comprise any material which can be coated with Si or SiO₂. The substrate can for example be selected from glass, silicon, SiO₂, semiconductors, metals, polymers, etc. In particular for optical applications, transparent substrates are preferred, but they are not relevant in biomedical applications.

For example, the primary substrate surface can be provided with a silicon layer with a thickness of, preferably, 50-500 nm by means of chemical vapour deposition or plasma deposition, or by another method known in the prior art.

Then the oxidation follows, e.g. with oxygen plasma or with another suitable oxidation agent, in order to obtain a SiO₂ layer on the primary substrate surface.

According to the invention, it is preferred, but not absolutely necessary that the covering of the substrate surface in step b) takes place with nanoparticles by means of a micellar diblock copolymer nanolithography technology, as described e.g. in EP 1 027 157 B1 and DE 197 47 815 A1. In micellar nanolithography, a micellar solution of a block copolymer is deposited onto a substrate, e.g. by means of dip coating, and under suitable conditions forms an ordered film structure of chemically different polymer domains on the surface, which inter alia depends on the type, molecular weight and concentration of the block copolymer. The micelles in the solution can be loaded with inorganic salts which, following deposition with the polymer film, can be oxidized or reduced to inorganic nanoparticles. A further development of this technology, described in the patent application DE 10 2007 017 032 A1, enables to two-dimensionally set both the lateral separation length of the polymer domains mentioned and thus also of the resulting nanoparticles and the size of these nanoparticles by means of various measures so precisely that nanostructured surfaces with desired spacing and/or size gradients can be manufactured. Typically, nanoparticle arrangements

manufactured with such a micellar nanolithography technology have a guasi-hexagonal pattern.

Fundamentally, the material of the nanoparticles is not particularly limited and may comprise any material known in the prior art for such nanoparticles . Typically, this is a metal or metal oxide. A broad spectrum of suitable materials is mentioned in DE 10 2007 014 538 A1. Preferably, the material of the metal or the metal component of the nanoparticles is selected from the group made up of Au, Pt, Pd, Ag, In, Fe, Zr, Al, Co, Ni, Ga, Sn, Zn, Ti, Si and Ge, mixtures and composites thereof. Specific examples for a preferred metal oxide are titanium oxide, iron oxide and cobalt oxide. Preferred examples for a metal are chromium, titanium, noble metals, e.g. gold, palladium and platinum, and gold is particularly preferred.

The term “particle” as used here also comprises a “cluster”, particularly as described and defined in DE 10 2007 014 538 A1 and DE 197 47 815 A1 and both terms can be used here interchangeably.

The enlargement of the metal nanoparticles by currentless deposition of elemental metal on the nanoparticles in step c) includes a reduction of the corresponding metal salt. A chemical agent, e.g. hydrazine or another suitable chemical reduction agent, or high-energy radiation, such as electron radiation or light (as described in DE 10 2009 053 406.7), can be used as a reduction agent.

The method, according to the invention, in the etching step d) can comprise one or several treatments with the same etching agent and/or with different etching agents. The etchant can basically be any etchant known in the prior art and suitable for the respective substrate surface. Preferably, the etchant is selected from the group of chlorine gases, e.g. Cl₂, BCl₃ and other gaseous chlorine compounds, fluorinated hydrocarbons, e.g. CHF₃, CH₂F₂, CH₃F, fluorocarbons, e.g. CF₄, C₂F₈, oxygen, argon, SF₆ and mixtures thereof. In a particularly preferred embodiment, CHF₃ is used in combination with SF₄ in at least one treatment step as etchant.

The duration of the entire etching treatment typically lies in the range of 10 s to 60 minutes, preferably 1 to 15 minutes .

Typically in step d), a plasma etching method (“reactive ion etching”) as described in DE 10 2007 014 538 A1 and Lohmüller et al. (NANO LETTERS 2008, Vol. 8, No. 5, 1429-1433) is used and preferably a mixture of CHF₃ with CF₄ is used.

Also good results are achieved if SF₆ is used as etchant or etchant component in at least one treatment step. In this way, very high etching rates can be achieved; however, the duration of the etching treatment must be carefully monitored, so that the etching process does not go too far and the desired metal-coated nanostructures remain preserved.

Typically, the obtained nanostructures have a diameter in the range of 10-100 nm, preferably 10-30 nm, and a height of 10-500 nm, preferably 10-150 nm. In the case of conical structures, the diameter data refer to the thickness at half height. The average spacings of the nanostructures are preferably in a range from 15 to 200 nm.

For some applications it is preferred that the nanoparticles used as an etching mask have a predetermined two-dimensional geometric arrangement on the substrate surface. Such arrangement has predetermined minimum or average particle spacings as a characteristic, wherein these predetermined particle spacings can be the same in all regions of the substrate surface or various regions can have different predetermined particle spacings. A geometric arrangement of this type can fundamentally be realized with any suitable method of the prior art, micellar nanolithography in particular, as explained in more detail above.

The nanostructures obtained after the etching step are functionalized, preferably, with at least one binding molecule, which enables or facilitates the binding of biological structures, molecules, microorganisms or cells.

Preferably, the binding molecule is a molecule, which specifically binds to surface structures of cells or to components of the extracellular matrix, or a molecule which can be received later by the cells cultivated in the substrate.

In more specific embodiments, the binding molecule is selected from the group of proteins or low-molecular peptides, in particular antibodies and fragments thereof, as well as enzymatically active proteins or domains thereof, lectins, carbohydrates, proteoglycans, glycoproteins, nucleic acids such as ssDNA, dsDNA, RNA, siRNA, lipids or glycolipids.

In a specific embodiment, the nanostructures are chemically functionalized with at least one binding molecule selected from molecules which bind to cell adhesion receptors (CAM) of cells, to specific receptors or binding sites on viruses, proteins or nucleic acids.

More specifically, these molecules are molecules which bind to the cell adhesion receptors of the cadherin, immunoglobulin superfamily (Ig-CAMS), selectin and integrin groups, in particular to integrins. In a still more specific embodiment, the binding molecule is selected from fibronectin, laminin, fibrinogen, tenascin, VCAM-1, MadCAM-1, collagen or a fragment thereof which binds specifically to cell adhesion receptors, in particular integrins, or a derivative thereof which binds specifically to cell adhesion receptors. Also signal-generating molecules, such as, for example, the entire receptor families of EGFR, FGFR and Notch/Jagged-1, can be addressed with these molecules.

However, the person skilled in the art would easily realize that variations of these molecules, as well as any other molecules with specific binding properties for certain target objects, in particular antibodies and other representatives of the above-mentioned substance classes, can also be used.

The functionalization takes place by immobilization of the binding molecule on the metal coating of the nanostructures. Methods for immobilization of binding molecules on metal substrates, in particular gold nanoparticles, are in principle known and are described, for example, in Arnold et al., ChemPhysChem (2004) 5, 383-388, Wolfram et al., Biointerphases 2007, Mar;2(1) :44-8, Ibii et al., Anal Chem. 2010, May 15; 82(10):4229-35, Sakata et al., Langmuir. 2007, Feb 27;23(5):2269-72 and Mateo-Marti et al., Langmuir. 2005, Oct 11;21(21):9510-7.

The three-dimensional nanostructures used according to the invention can be biofunctionalized at room temperature typically within half an hour and are thus clearly superior, with respect to the time and cost outlays, compared to the three-dimensional microstructures of the prior art described in the introduction to the present text.

Some fundamental methods for immobilization of the preferred binding molecules, e.g. antibodies, peptides, recombinant proteins, glycoproteins, nucleic acids or native proteins, on metal substrates are discussed briefly below.

The orientation-specific immobilization of recombinant proteins is possible, for example, with Ni-NTA-complex reactions (Wolfram et al., above). Furthermore, all proteins and antibodies can be covalently bound with the help of DTSSP and related thiol-based linkers on gold and silver nanoparticles (see Example 2). Immobilization of antibodies or fragments thereof through immobilization of protein A/G or L is also possible. The bioactive molecules can be bound directly or indirectly through linker systems. Chemisorption, affinity-based as well as protein-mediated immobilizations can be used.

Suitable conditions for obtaining column-like nanostructures on a substrate surface, coated with SiO₂, and for their functionalization are described in the exemplary embodiments in more detail. It will become clear for the person skilled in the art, however, that variations of these conditions as a function of the specific materials used may be reguired and can be determined without difficulty by means of routine experiments.

The substrate surfaces with a three-dimensional nanostructure, obtained with the method according to the invention, offer possibilities for diverse applications in the areas of semiconductor technology, biology, medicine, pharmacy, sensor technology and medical technology, in particular for bioactive and biointelligent surfaces or implant surfaces as well as tissue engineering.

The functionalized nanostructured substrate surfaces are suitable, in particular, for identification of biological target structures, molecules, microorganisms or cells in a sample and/or for their isolation from it. For example, the sample can be a body fluid, in particular blood, interstitial or mucosal fluids, or a solid tissue sample. The target structures can be molecules, which are known as diagnostic markers, or the target cells can be, for example, certain tumour cells, trophoblasts or other desired cell types or components thereof.

An essential aspect of the invention is related to a device for specific binding of biological target structures, molecules, microorganisms or cells, which are present in a sample, in particular a sample as defined above, which comprises such a nanostructured substrate surface.

In a specific embodiment, this device is a component part of a probe, which is designed so that it can be introduced in a living organism/body and brought in contact with its body fluids.

In a particularly preferred embodiment, the device is characterized in that at least one part of the probe has the shape of a needle and can be introduced into the blood stream of a living organism. In this way, for example, certain circulating cell types can be isolated in a targeted manner from the blood and then identified. Here, the dimensions of the needle are preferably within the ranges known for the needles and cannulas (e.g. for injections and withdrawal of blood samples) used in medical applications and can be easily optimized by means of routine tests.

Since both the physical parameters of a nanostructured substrate surface according to the invention can be adjusted by varying the height, thickness, form and spacing of the nanostructures, and the chemical parameters can be adjusted flexibly and precisely by the selection of special metal coatings and immobilized binding molecules, specific surfaces can be created, which ensure not only an optimal adhesion of the target molecules as well as the cells (which increases, correspondingly, the detection sensitivity), but, in addition, permit also to exert influence on the behaviour of the live cells themselves, since the cells, as it is known, perceive not only chemical but also structural signals, such as the topography of a substrate surface.

BRIEF DESCRIPTION OF THE FIGURES

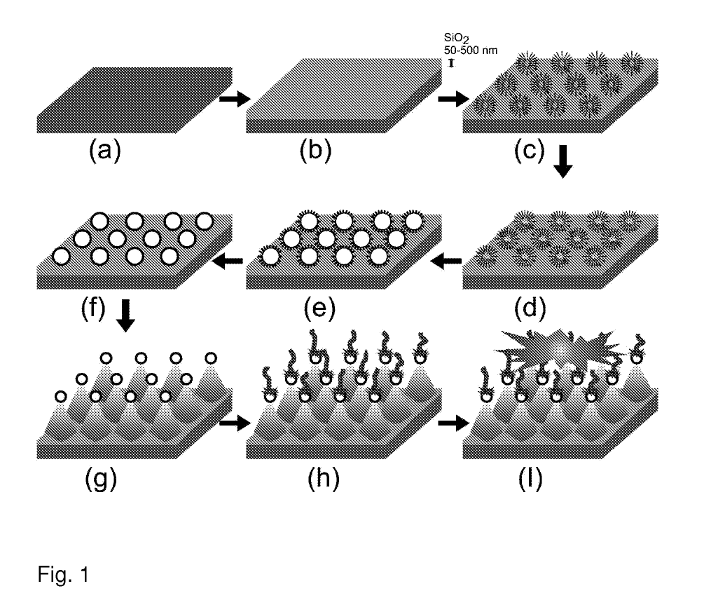

FIG. 1 shows schematically the main steps of the method according to the invention.

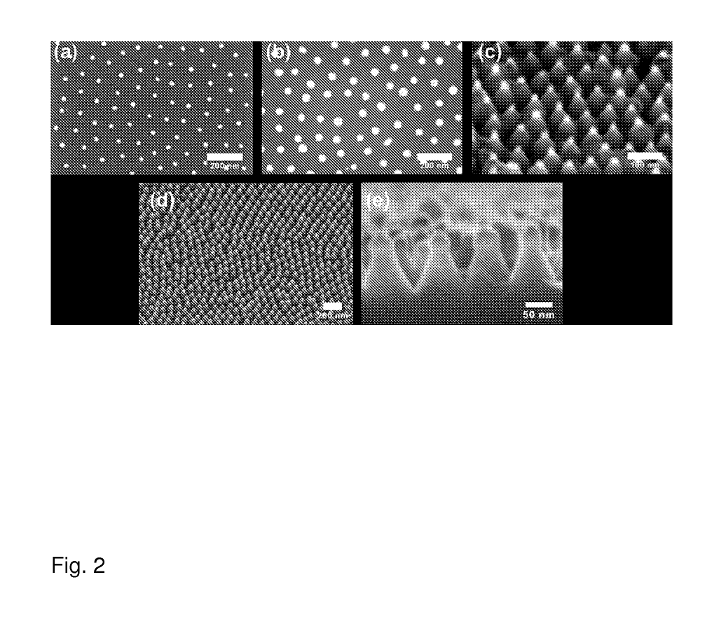

FIG. 2 shows scanning electron microscope images of a substrate surface in different phases of the method according to the invention:
(a) after the application of gold nanoparticles by means of micellar block nanolithography;
(b) after enlargement of the gold nanoparticles by means of currentless deposition;
(c) with metal-coated column-like structures after the etching;
(d) shows the large-range order in the urn range;
(e) shows a lateral view of the conical pillars.

The following examples are used for more in depth explanation of the present invention, without limiting the same thereto, however.

### EXAMPLE 1

Generation of Column-Like Nanostructures on a Substrate with an Arrangement of Gold Nanoparticles

### 1. Providing the Substrate Surface

First a primary substrate surface was provided with a 50-500 nm thick silicon layer by chemical vapour deposition or plasma deposition. Then activation was performed in oxygen plasma (150 W, 0.1 mbar, 30 minutes) in order to obtain a SiO₂ layer on the primary substrate surface (FIG. 1b).

2. Coating with Gold Nanoparticles

The SiO₂ substrate surface formed in the first step was coated with gold nanoparticles in a defined arrangement by means of micellar nanolithography (FIG. 1c). In this step, one of the protocols described in EP 1 027 157 B1, DE 197 47 815 A1 or DE 10 2007 017 032 A1 can be followed. The method involves the deposition of a micellar solution of a block copolymer (e.g. polystyrene(n)-b-poly(2-vinylpyridine(m)) in toluene) onto the substrate, e.g. by means of dip coating, as a result of which an ordered film structure of polymer domains is formed on the surface. The above-described activation step in oxygen plasma stimulates adhesion of the micelles on the surface.

The micelles in the solution are loaded with a gold salt, preferably HAuCl₄ which, following deposition with the polymer film, is reduced to the gold nanoparticles. To this purpose, a brief hydrogen plasma activation (200 W, 0.5 mbar, 1 minute) was performed in order to obtain gold particle germs in the micelle cores (FIG. 1d).

### 3. Enlargement of Gold Nanoparticles by Means of Currentless Deposition

The currentless deposition took place by immersing the surface in a solution of 0.1% HAuCl₄ and 0.2 mM NH₃OHCl (1:1) for 3.5 minutes. Under these reducing conditions, the gold salt in the solution is reduced to elemental gold which is deposited selectively on the gold particle germs and enlarges them (FIG. 1e). Now the polymer micelles can be removed from the surface and this is achieved by exposing the surface to hydrogen plasma (150 W, 0.4 mbar, 45 minutes). At this point in time, the substrate surface is decorated with a guasi-hexagonal two-dimensional arrangement of gold nanoparticles with a desired size (FIG. 1f).

### 4. Etching Step

Subsequently, the etching of the SiO₂ layer covered with gold nanoparticles took place to a desired depth. A “reactive ion etcher” from Oxford Plasma, device: PlasmaLab 80 plus was used to this end. Other devices known in the prior art are likewise fundamentally suitable, however.

The etching was performed with a mixture of process gases CHF₃ and CF₄ (10:1) at a total pressure of 10 mTorr, temperature of 20° C. and energy of 30 W. The duration of the etching treatment varied depending on the desired depth of the etching within about 1-15 minutes. As a result, column-like or obtuse-conical nanostructures were obtained, which still showed gold nanoparticles on their upper side (FIG. 1g).

### EXAMPLE 2

### Functionalization of the Nanostructures

For the functionalization of the three-dimensional nanostructures obtained in Example 1, various protocols were used.

(Protocol A) The presented nanostructures were incubated in PBS for 30 min at room temperature or for 2 h at 4° C. with 20-60 μl 0.25-5 mM DTSSP (3,3′-dithiobis[sulfosuccinimidyl-propionate], Thermo Fisher Scientific, Rockford USA) and subsequently washed with PBS several times. Then every substrate was incubated for 2 h at 4° C. or for 30 min at room temperature with the desired antibody (c=10 μg/ml) and subsequently washed with PBS. In the case when the antibody solution contains Tris Buffer or glycine, the antibody should be dialysed against PBS prior to the incubation. Besides the thiol chemistry-based chemisorption, affinity immobilizations were also used.

(Protocol B) Gold-doped substrate surfaces were incubated for two hours with thiolated nitrilotriacetic acid (NTA) in ethanol at room temperature. Subsequently nickel was bound as NiCl₂ (10 mM in HBS) to the NTA by means of 15 minutes of incubation. After rebuffering, incubation was performed with a protein solution (His-tag protein 10 μg/ml in PBS) for 4 to 12 hours at 4° C. Finally, the substrates were washed.

(Protocol C) Another protocol is the direct immobilization of proteins by means of chemisorption. Here, protein A, G or L was heated for 5 minutes at 65° and subsequently incubated under slightly basic buffer conditions (Tris-HCl pH 8-9.5) for one hour on the substrates.

(Protocol D) The so produced substrates were used for antibody binding. Here, an antibody solution (1-2 mg/mL in PBS) 1:50 was diluted in PBS and subseguently incubated for two hours at room temperature. Finally, the substrates were briefly washed.

(Protocol E) Besides the immobilization of peptides and proteins, also nucleic acids were immobilized. Here, thiolated ssDNA fragments (100 pMol in water) were incubated on the substrates for four hours at 4° and subseguently washed. The complementary ssDNA strand (100 pMol in water) was incubated for one hour at 37° C. on the substrates. The successful binding was proved by a fluorescent group in the second ssDNA strand.

The functionalized substrate surfaces (FIG. 1h) can now be used for binding of target structures, in particular target cells (FIG. 1i).

1. A method for generating on substate surfaces nanostructures with a column-like or conical shape, which have a metal coating on an upper side thereof, said method comprising the steps of:
a) providing a substrate surface coated with SiO₂ or consisting of SiO₂;
b) covering the substrate surface with an arrangement of metal nanoparticles;
c) contacting the substrate surface with a metal salt solution under reducing conditions,
whereby a reduction of the metal salt and currentless deposition of elemental metal on the metal nanoparticles and a corresponding enlargement of the metal nanoparticles is caused; and
d) etching to a depth of 10-500 nm of the substrate surface covered with the nanoparticles obtained in step c), wherein the nanoparticles act as an etching mask and etching parameters are adjusted in such a way that column-like structures or conical structures are formed underneath the nanoparticles and the nanoparticles remain kept there as a structure coating.

2. The method according to claim 1, wherein the etching step comprises a treatment with an etchant which is selected from the group consisting of chlorine, gaseous chlorine compounds, fluorinated hydrocarbons, fluorocarbons, oxygen, argon, SF₆ and mixtures thereof.

3. The method according to claim 1, wherein the etching step is performed for a period of time in a range from 10 s to 60 min.

4. The method according to claim 1, wherein the nanoparticles in step b) have a predetermined two-dimensional geometric arrangement.

5. The method according to claim 1, wherein the metallic nanoparticles in step b) are applied to the substrate surface by micellar nanolithography.

6. The method according to claim 1, wherein the nanoparticles comprises metals or metal oxides.

7. The method according to claim 6, wherein the nanoparticles comprise a member selected from the group consisting of Au, Pt, Pd, Ag, In, Fe, Zr, Al, Co, Ni, Ga, Sn, Zn, Ti, Si, Ge, mixtures and composites thereof.

8. The method according to claim 7, wherein the nanoparticles are noble metal nanoparticles.

9. The method according to claim 1, further comprising functionalizing the metal coating of the nanostructures obtained with the steps a)-d) with a binding molecule, which enables or facilitates binding of biological structures, molecules, microorganisms or cells.

10. The method according to claim 9, wherein the binding molecule is a molecule that binds specifically on surface structures of cells or components of an extracellular matrix.

11. The method according to claim 9, wherein the binding molecule is a member selected from the group consisting of proteins, low-molecular weight peptides, lectins, carbohydrates, proteoglycans, glycoproteins, nucleic acids, lipids and glycolipids.

12. The method according to claim 1, wherein the step a) comprises the steps i) coating a substrate surface with a 50-500 nm thick Si layer and ii) oxidizing the Si layer, whereby the substrate surface coated with SiO₂ of step a) is provided.

13. A substrate surface comprising column-like or conical nanostructures, which can be obtained with the method according to claim 1.

14. The substrate surface according to claim 13, wherein the column-like structures or conical structures have a height of 10-500 nm, a thickness of 10-100 nm, as well as an average spacing from 15 to 200 nm, and the metal coating of nanopillars/nanocones is formed from noble metal nanoparticles.

15. The substrate surface according to claim 13, which is adapted for use in semiconductor technology, optics, biology, medicine, pharmacy, sensor technology, medical engineering or tissue engineering.

16. A method of using the substrate surface according to claim 13 for identification of biological target structures, molecules, microorganisms or cells in a sample and/or their isolation therefrom.

17. The method according to claim 16, wherein the sample is a body fluid, interstitial fluid or mucosa fluid, or a solid tissue sample.

18. A device for specific binding of biological target structures, molecules, microorganisms or cells, which are present in a sample, comprising a substrate surface according to claim 13.

19. The device according to claim 18, wherein the device is a component part of a probe, which is designed in such a way that the probe can be introduced in a living organism and can be brought in contact with body fluids of the living organism.

20. The device according to claim 19, wherein at least one part of the probe has a form of a needle and can be introduced in a blood stream of a living organism.
